(19) United States
(12) Patent Application Publication
(10) Pub. No.: US 2024/0335663 A1
(43) Pub. Date: Oct. 10, 2024

(54) SYSTEM AND METHOD FOR LEAD PLACEMENT BY EVOKED POTENTIAL

(71) Applicant: Boston Scientific Neuromodulation Corporation, Valencia, CA (US)

(72) Inventors: G. Karl Steinke, Valencia, CA (US); Soroush Niketeghad, Stevenson Ranch, CA (US); Tomasz Mark Fraczek, Yucaipa, CA (US)

(21) Appl. No.: 18/624,644

(22) Filed: Apr. 2, 2024

Related U.S. Application Data
(60) Provisional application No. 63/457,725, filed on Apr. 6, 2023.

(51) Int. Cl. A61N 1/36; A61N 1/02; A61N 1/05; A61N 1/372

(52) U.S. Cl. CPC A61N 1/3614; A61N 1/025; A61N 1/36178; A61N 1/37247; A61N 1/0534

(57) ABSTRACT

This document discusses a computer-implemented method of operating a neurostimulation device to deliver electrical neurostimulation when connected to an implantable stimulation lead. The method includes delivering neurostimulation to a subject that produces evoked potential signals, sensing the evoked potential signals resulting from the neurostimulation, detecting changes in the sensed evoked potential signals in response to a change in at least one of the number of repeated presentations of the neurostimulation or a change in a parameter of the first neurostimulation, and producing an indication of proximity of the stimulation lead to a preferred lead location according to the detected changes in the evoked potential signals.

### CLAIM OF PRIORITY

This application claims the benefit of U.S. Provisional Application No. 63/457,725 filed on Apr. 6, 2023, which is hereby incorporated by reference in its entirety.

### TECHNICAL FIELD

This document relates generally to medical devices and more particularly to a system for neurostimulation.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device can be used to program the implantable neurostimulator with stimulation parameters controlling the delivery of the neurostimulation energy.

In one example, the neurostimulation energy is delivered in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Neurostimulation systems may offer many programmable options for the parameters of the neurostimulation to customize the neurostimulation therapy for a specific patient. For some types of neurostimulation (e.g., DBS) the efficacy of the neurostimulation for the patient may depend on an intricate balance of stimulation location coupled with the programmed stimulation waveform. Finding the right stimulation location can be a complicated and time consuming process, and the number of programmable options can create an extensive parameter search space for the physician or clinician. Finding the optimal neurostimulation for a particular patient may take a lot of time in the clinic for both the clinic staff and the patient.

### SUMMARY

Electrical neurostimulation energy can be delivered in the form of electrical neurostimulation pulses to treat a neurological condition of the patient. The pulses can be delivered using an implantable stimulation lead. The lead can have multiple electrodes and may be configurable into many electrode configurations. Proper placement of the stimulation lead produces the intended benefit to the patient. Improper lead placement may result in less benefit to the patient and may require a subsequent procedure to reset the neurostimulation and the lead.

Example 1 includes subject matter (such as a method of operating a neurostimulation device to deliver electrical neurostimulation when the neurostimulation device is connected to an implantable stimulation lead) comprising delivering first neurostimulation to a subject that produces evoked potential signals; delivering second neurostimulation that includes at least one of multiple presentations of the first neurostimulation or a change in a parameter of the first neurostimulation; sensing the evoked potential signals resulting from the first neurostimulation and the second neurostimulation; detecting changes in the sensed evoked potential signals between the first neurostimulation and the second neurostimulation; and producing an indication of proximity of the stimulation lead to a preferred lead location according to the detected changes in the sensed evoked potential signals.

In Example 2, the subject matter of Example 1 optionally includes delivering second neurostimulation that includes multiple neurostimulation pulses; detecting a change in a signal feature of the sensed evoked potential signals between neurostimulation pulses; and determining proximity of the stimulation lead according to a degree of saturation in the sensed evoked potential signals.

In Example 3, the subject matter of one or both of Examples 1 and 2 optionally includes changing an amplitude of the neurostimulation; detecting a change in one or more features of the sensed evoked potential signals; and determining proximity of the stimulation lead according to a degree of saturation in the sensed evoked potential signals.

In Example 4, the subject matter of one or any combination of Examples 1-3 optionally includes delivering multiple bursts of neurostimulation pulses, detecting a change in magnitude of a feature of the post-burst evoked potential signal over the multiple bursts, and determining proximity of the stimulation lead according to the detected change in magnitude of the post-burst evoked potential signal.

In Example 5, the subject matter of one or any combination of Examples 1-4 optionally includes delivering multiple bursts of neurostimulation pulses, sensing a post-burst evoked potential signal after a burst of neurostimulation pulses, detecting a phase change of the post-burst evoked potential signal, and producing the indication according to the detected phase change.

In Example 6, the subject matter of one or any combination of Examples 1-5 optionally includes delivering multiple bursts of neurostimulation pulses, sensing a post-burst evoked potential signal after a last pulse of a burst of neurostimulation pulses, detecting changes in timing of a feature of the post-burst evoked potential signal over the multiple bursts, and producing the indication according to the detected changes in timing of the feature of the post-burst evoked potential signal.

In Example 7, the subject matter of one or any combination of Examples 1-6 optionally includes presenting an indication that the stimulation lead is close to the neurostimulation target according to a degree of saturation in the sensed evoked potential signals, presenting an indication that the stimulation lead is off of the preferred lead location according to a monotonical increase in amplitude of the sensed evoked potential signals, and presenting an indication that the stimulation lead is not close to the preferred lead location according to an amplitude of the sensed evoked potential signals that increases at first slower rate and then increases at second faster rate.

In Example 8, the subject matter of one or any combination of Examples 1-7 optionally includes presenting an indication that the stimulation lead is close to the preferred lead location when the sensed evoked potential signals saturate at a first signal amplitude at a first time relative to the neurostimulation, presenting an indication that the stimulation lead is not close to the preferred lead location when the sensed evoked potential signals saturate at a second signal amplitude at a second time relative to the neurostimulation, wherein the second signal amplitude is larger than the first signal amplitude and the second time is later than the first time relative to the neurostimulation.

Example 9 includes subject matter (such as a neurostimulation system) comprising a stimulation circuit configured to deliver electrical neurostimulation to a subject when coupled to an implantable stimulation lead, a sensing circuit configured to sense evoked potential signals when coupled to the stimulation lead, a control circuit operatively coupled to the stimulation circuit and the sensing circuit, and configured to initiate delivery of neurostimulation to the subject that produces an evoked potential signal and record sensed evoked potential signals resulting from the neurostimulation, and signal processing circuitry configured to detect changes in the sensed evoked potential signals in response to a change in at least one of the number of repeated presentations of the neurostimulation or a change in a parameter of the first neurostimulation, and produce an indication of proximity of the stimulation lead to a preferred lead location according to the detected changes in the evoked potential signals.

In Example 10, the subject matter of Example 9 optionally includes a control circuit configured to initiate delivery of multiple neurostimulation pulses by the stimulation circuit, and a signal processing circuit configured to detect a change in a signal feature of the sensed evoked potential signals between neurostimulation pulses, and produce the indication of proximity of the stimulation lead according to a degree of saturation in the sensed evoked potential signals.

In Example 11, the subject matter of one or any combination of Examples 9 and 10 optionally includes a control circuit configured to initiate delivery of multiple neurostimulation pulses by the stimulation circuit, and change an amplitude of the neurostimulation pulses, and a signal processing circuit configured to measure amplitude of the sensed evoked potential signals with the changing amplitude of the neurostimulation, and produce the indication of proximity of the stimulation lead according to whether the sensed evoked potential signals show saturation.

In Example 12, the subject matter of one or any combination of Examples 9-11 optionally includes a control circuit configured to initiate delivery of multiple bursts of neurostimulation pulses and record a sensed a post-burst evoked potential signal after a last pulse of a burst of neurostimulation pulses, and a signal processing circuit configured to detect a change in magnitude of a feature of the post-burst evoked potential signal over the multiple bursts and produce the indication of proximity of the stimulation lead according to the detected change in magnitude of the post-burst evoked potential signal.

In Example 13, the subject matter of one or any combination of Examples 9-12 optionally includes a control circuit configured to initiate delivery of multiple bursts of neurostimulation pulses and record a post-burst evoked potential signal sensed after at least a last pulse of a burst of neurostimulation pulses, and a signal processing circuit configured to measure frequency of the post-burst evoked potential signal over the multiple bursts and produce the indication of proximity of the stimulation lead according to detected changes in frequency of the post-burst evoked potential signal.

In Example 14, the subject matter of one or any combination of Examples 9-13 optionally includes a control circuit configured to initiate delivery of multiple bursts of neurostimulation pulses and record a sensed a post-burst evoked potential signal after at least a last pulse of a burst of neurostimulation pulses, and a signal processing circuit configured to detect a feature in the post-burst evoked potential signal, detect a change in timing of the detected feature over the multiple bursts of neurostimulation pulses, and produce the indication of proximity of the stimulation lead according to detected changes in timing of the feature of the post-burst evoked potential signal.

In Example 15, the subject matter of one or any combination of Examples 9-14 optionally includes a user interface operatively coupled to a signal processing circuit configured to detect saturation of a feature in a sensed evoked potential signal and present an indication on the user interface that the stimulation lead is close to the preferred lead location according to a degree of saturation detected in the sensed evoked potential signals, detect when a magnitude of the sensed evoked potential signal increases monotonically with the neurostimulation and present an indication on the user interface that the stimulation lead is off the preferred lead location when detecting the monotonical increase in the sensed evoked potential signal, and detect when a rate of increase in the magnitude of the feature in the sensed evoked potential signal changes and present an indication on the user interface that the stimulation lead is not close to the preferred lead location according to the detected change in rate of increase in the magnitude of the feature.

In Example 16, the subject matter of one or any combination of Examples 9-15 optionally includes a user interface operatively coupled to a signal processing circuit configured to detect when a sensed evoked potential signal saturates at a first signal amplitude at a first time relative to delivery of the neurostimulation and present an indication on the user interface that the stimulation lead is close to the preferred lead location in response to the detection of the first signal amplitude at the first time, and detect when a sensed evoked potential signal saturates at a second signal amplitude larger than the first signal amplitude at a second time relative to the neurostimulation later than the first time and present an indication on the user interface that the stimulation lead is not close to the preferred lead location in response to the detection of the second signal amplitude at the second time.

In Example 17, the subject matter of one or any combination of Examples 9-16 optionally includes a communication circuit configured to communicate information with a separate device, and the stimulation circuit, sensing circuit, and communication circuit being included in an implantable pulse generator.

In Example 18, the subject matter of one or any combination of Examples 9-17 optionally includes a user interface operatively coupled to the signal processing circuit and a sensing circuit configured to configured to sense an evoked potential signal for each electrode of multiple electrodes of the stimulation lead. The signal processing circuit is configured to detect the changes in the sensed evoked potential signals, and simultaneously present the changes of the sensed evoked potential signal for each electrode on the user interface.

Example 19 includes subject matter (or can optionally be combined with one or any combination of Examples 1-18 to include such subject matter) comprising a computer readable storage medium including instructions that when performed by processing circuitry of a neurostimulation system, cause the neurostimulation system to perform actions including initiate delivery of neurostimulation to produce an evoked potential signal in a subject, record sensed evoked potential signals resulting from the neurostimulation, detect changes in the sensed evoked potential signals in response to a change in at least one of the number of repeated presentations of the neurostimulation or a change in a parameter of the first neurostimulation, and display an indication of proximity of the stimulation lead to a preferred lead location according to the detected changes in the sensed evoked potential signals.

In Example 20, the subject matter of Example 19 optionally includes the computer readable storage medium further including instructions that cause the neurostimulation system to perform actions including detect saturation in a sensed evoked potential signal and display an indication on the user interface that the stimulation lead is close to the preferred lead location in response to detecting the saturation, detect when amplitude of a sensed evoked potential signal increases monotonically with the neurostimulation and display an indication on the user interface that the stimulation lead is off the preferred lead location in response to detecting the monotonical increase in the sensed evoked potential signal, and detect when a rate of increase in amplitude of a sensed evoked potential signal changes and display an indication on the user interface that the stimulation lead is not close to the preferred lead location in response to detecting the change in rate of increase in amplitude.

These non-limiting examples can be combined in any permutation or combination. This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of portions of an example of a neurostimulation system.

FIG. 2 is an illustration of portions of another example of a neurostimulation system.

FIG. 3 is an illustration of an example of an implantable pulse generator (IPG) and an implantable lead system.

FIG. 4 is an illustration of another example of an IPG and an implantable lead system.

FIG. 5 is a schematic side view of an example of an electrical stimulation lead.

FIGS. 6A-6H are illustrations of an example of electrodes of a stimulation lead.

FIG. 7 is a block diagram of portions of an example of a medical device for providing neurostimulation.

FIG. 8 is a flow diagram of a method to operate a neurostimulation system.

FIG. 9 is an illustration of the variation in a sensed neural response with the distribution of the electrodes at different levels of the stimulation lead.

FIG. 10 is an illustration of waveforms of a burst of pulses of neurostimulation and the evoked potential signal.

FIG. 11 is an illustration of delivering bursts of neurostimulation pulses with changing amplitude of the pulses.

FIG. 12 is an illustration of the results of delivering neurostimulation pulses with increasing amplitude.

FIG. 13 is an illustration of waveforms of multiple bursts of neurostimulation pulses and evoked response signals resulting from the bursts of neurostimulation pulses.

FIG. 14 is an illustration of waveforms of post-burst evoked potential signals.

FIG. 15 is a graph showing percentage change in magnitude of a feature of a sensed evoked signal as a function of the number of presentations or repetitions of the evoking neurostimulation.

FIGS. 16A and 16B are illustrations of waveforms of repetitions of a burst of neurostimulation pulses and the sensed evoked response signals resulting from the repeating bursts of neurostimulation pulses.

FIGS. 17A and 17B are graphs showing the change in magnitude of a feature of the sensed evoked signals in FIGS. 16A and 16B.

FIG. 18 is another illustration of waveforms of post-burst evoked potential signals.

FIG. 19 is a plot of small neural responses and large neural responses.

FIG. 20 is a flow diagram of an example of evaluating the position of a stimulation lead when the lead is connected to a neurostimulation system.

FIGS. 21-25 illustrate examples of displays of a user interface of a neurostimulation system.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses devices, systems and methods for programming and delivering electrical neurostimulation to a patient or subject. Advancements in neuroscience and neurostimulation research have led to a demand for delivering complex patterns of neurostimulation energy for various types of therapies. The present system may be implemented using a combination of hardware and software designed to apply any neurostimulation (neuromodulation) therapy, including but not being limited to DBS, SCS, PNS, FES, Occipital Nerve Stimulation (ONS), Sacral Nerve Stimulation (SNS), and Vagus Nerve Stimulation (VNS) therapies.

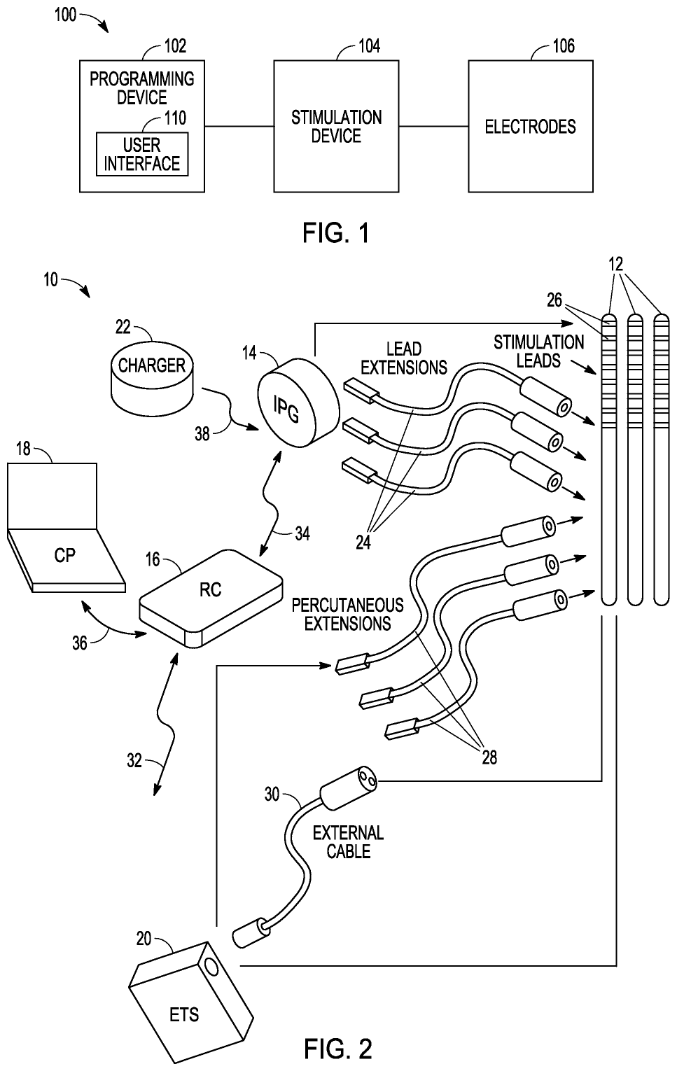

FIG. 1 illustrates an example of portions of a neurostimulation system 100. System 100 includes electrodes 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using multiple stimulation parameters, such as stimulation parameters specifying a pattern of the electrical pulses and a selection of electrodes through which each of the electrical pulses is delivered. In various embodiments, at least some of the stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device 104 via a wired or wireless link.

In this document, a “user” includes a physician or other clinician or caregiver who treats the patient using system 100; a “patient” includes a person who receives or is intended to receive neurostimulation delivered using system 100. In various embodiments, the patient can be allowed to adjust his or her treatment using system 100 to certain extent, such as by adjusting certain therapy parameters and entering feedback and clinical effect information.

In various embodiments, programming device 102 can include a user interface 110 that allows the user to control the operation of system 100 and monitor the performance of system 100 as well as conditions of the patient including responses to the delivery of the neurostimulation. The user can control the operation of system 100 by setting and/or adjusting values of the user-programmable parameters.

In various embodiments, user interface 110 can include a graphical user interface (GUI) that allows the user to set and/or adjust the values of the user-programmable parameters by creating and/or editing graphical representations of various stimulation waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields.

In various embodiments, system 100 can be configured for neurostimulation applications. User interface 110 can be configured to allow the user to control the operation of system 100 for neurostimulation. For example, system 100 as well as user interface 110 can be configured for DBS applications. The DBS configurations include various features that may simplify the task of the user in programming the stimulation device 104 for delivering DBS to the patient, such as the features discussed in this document.

FIG. 2 is an illustration of portions of another example of a neurostimulation system 10 that includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22. The IPG 14 can optionally be physically connected via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The IPG 14 can be implanted into a patient's body, for example, below the patient's clavicle area or within the patient's buttocks or abdominal cavity. The implantable pulse generator can have multiple stimulation channels (e.g., 8 or 16) which may be independently programmable to control the magnitude of the current stimulus from each channel. The IPG 14 can have one, two, three, four, or more connector ports, for receiving the terminals of the leads 12.

The ETS 20 may also be physically connected, optionally via the percutaneous lead extensions 28 and external cable 30, to the stimulation leads 12. The ETS 20, which may have similar pulse generation circuitry as the IPG 14, can also deliver electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a wireless communications link 32. Once the IPG 14 and neurostimulation leads 12 are implanted, the RC 16 may be used to telemetrically communicate with or control the IPG 14 via communications link 34. The communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

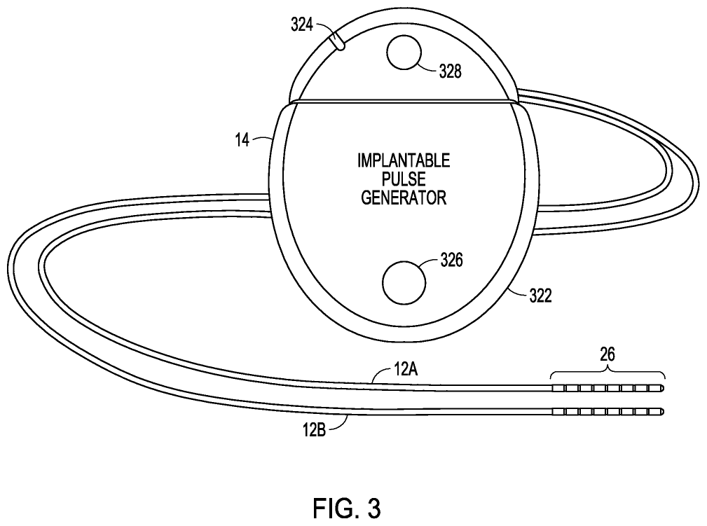

FIG. 3 is an illustration of an example of an IPG 14 (e.g., IPG 14 in FIG. 2) and an implantable lead system that includes stimulation leads (e.g., stimulation leads 12 in FIG. 2). The IPG 14 can be used as stimulation device 104 in FIG. 1. As illustrated in FIG. 3, IPG 14 that can be coupled to implantable leads 12A and 12B at a proximal end of each lead. The distal end of each lead includes electrical contacts or electrodes 26 for contacting a tissue site targeted for electrical neurostimulation. As illustrated in FIG. 3, leads 12A and 12B each include 8 electrodes 26 at the distal end. The number and arrangement of leads 12A and 12B and electrodes 26 as shown in FIGS. 2 and 3 are only examples, and other numbers and arrangements are possible. In various examples, the lead electrodes 26 are ring electrodes. In various examples the lead electrodes 26 include one or more segmented electrodes.

The IPG 14 can include a hermetically sealed IPG case 322 to house the electronic circuitry of IPG 14. IPG 14 can include an electrode 326 formed on IPG case 322. IPG 14 can include an IPG header 324 for coupling the proximal ends of leads 12A and 12B. IPG header 324 may optionally also include an electrode 328. One or both of electrodes 326 and 328 may be used as a reference electrode.

The implantable leads and electrodes may be configured by shape and size to provide electrical neurostimulation energy to a neuronal target included in the subject's brain. Neurostimulation energy can be delivered in a monopolar (also referred to as unipolar) mode using electrode 326 or electrode 328 and one or more electrodes selected from electrodes 26. Neurostimulation energy can be delivered in a bipolar mode using a pair of electrodes of the same lead (lead 12A or lead 12B). Neurostimulation energy can be delivered in an extended bipolar mode using one or more electrodes of a lead (e.g., one or more electrodes of lead 12A) and one or more electrodes of a different lead (e.g., one or more electrodes of lead 12B).

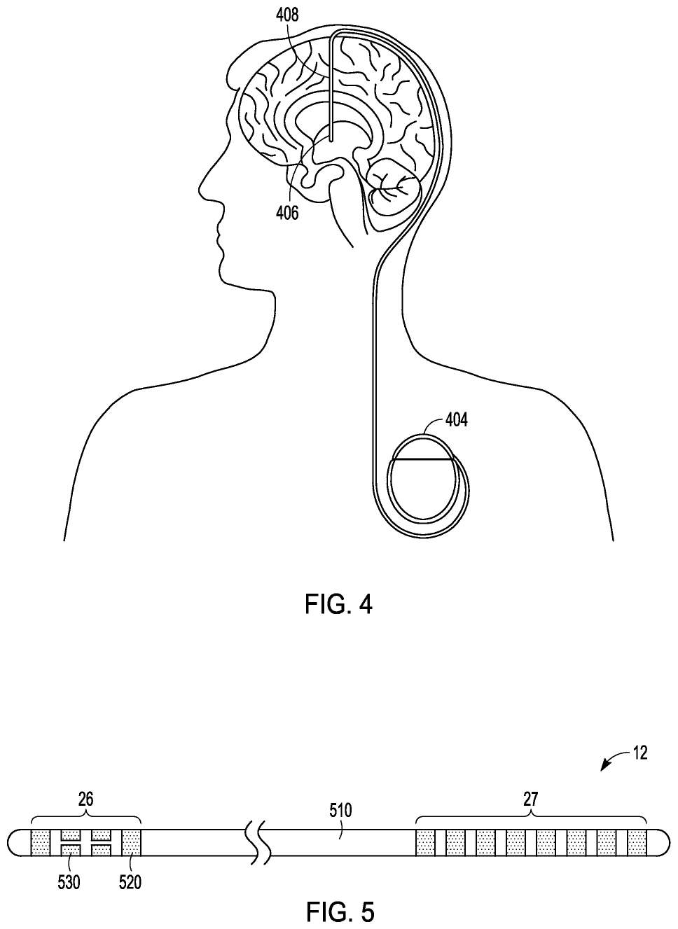

FIG. 4 illustrates another example of an IPG 404 and an implantable lead system 408 arranged to provide neurostimulation to a patient. An example of IPG 404 includes IPG 14 of FIGS. 2 and 3. An example of lead system 408 includes one or more of leads 12A and 12B in FIG. 3. The distal end 406 of the lead includes multiple electrodes (e.g., electrodes 26 in FIG. 3) In the illustrated embodiment, implantable lead system 408 is arranged to provide Deep Brain Stimulation (DBS) to a patient, with the stimulation target being neuronal tissue in a subdivision of the thalamus of the patient's brain. Other examples of DBS targets include neuronal tissue of the globus pallidus (GPi), the subthalamic nucleus (STN), the pedunculopontine nucleus (PPN), substantia nigra pars reticulate (SNr), cortex, globus pallidus externus (GPe), medial forebrain bundle (MFB), periaquaductal gray (PAG), periventricular gray (PVG), habenula, subgenual cingulate, ventral intermediate nucleus (VIM), anterior nucleus (AN), other nuclei of the thalamus, zona incerta, ventral capsule, ventral striatum, nucleus accumbens, and any white matter tracts connecting these and other structures.

After implantation, a clinician will program the neurostimulation device 400 using a CP 18, remote control, or other programming device. The programmed neurostimulation device 400 can be used to treat a neurological condition of the patient, such as Parkinson's Disease, Tremor, Epilepsia, Alzheimer's Disease, other Dementias, Stroke, Multiple Sclerosis, Amyotrophic Lateral Sclerosis (ALS), Autism, brain injury, brain tumor, migraine or other pain or headache condition, and any neurological syndromes that are congenic, degenerative, or acquired.

Returning to FIG. 3, the electronic circuitry of IPG 14 can include a stimulation control circuit that controls delivery of the neurostimulation energy. The stimulation control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

FIG. 5 is a schematic side view of an embodiment of an electrical stimulation lead. FIG. 5 illustrates a stimulation lead 12 with electrodes 26 disposed at least partially about a circumference of the lead 12 along a distal end portion of the lead and terminals 27 disposed along a proximal end portion of the lead. The lead 12 can be implanted near or within the desired portion of the body to be stimulated (e.g., the brain, spinal cord, or other body organs or tissues). In one example of operation for deep brain stimulation, access to the desired position in the brain can be accomplished by drilling a hole in the patient's skull or cranium with a cranial drill (commonly referred to as a burr), and coagulating and incising the dura mater, or brain covering. The lead 12 can be inserted into the cranium and brain tissue with the assistance of a stylet (not shown). The lead 12 can be guided to the target location within the brain using, for example, a stereotactic frame and a microdrive motor system. In some embodiments, the microdrive motor system can be fully or partially automatic. The microdrive motor system may be configured to perform one or more the following actions (alone or in combination): insert the lead 12, advance the lead 12, retract the lead 12, or rotate the lead 12.

In some embodiments, measurement devices coupled to the muscles or other tissues stimulated by the target neurons, or a unit responsive to the patient or clinician, can be coupled to the implantable pulse generator or microdrive motor system. The measurement device, user, or clinician can indicate a response by the target muscles or other tissues to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

The lead 12 for deep brain stimulation can include stimulation electrodes, recording electrodes, or both. In at least some embodiments, the lead 12 is rotatable so that the stimulation electrodes can be aligned with the target neurons after the neurons have been located using the recording electrodes. Stimulation electrodes may be disposed on the circumference of the lead 12 to stimulate the target neurons. Stimulation electrodes may be ring-shaped so that current projects from each electrode equally in every direction from the position of the electrode along a length of the lead 12. In the embodiment of FIG. 5, two of the electrodes 520 are ring electrodes 520. Ring electrodes typically do not enable stimulus current to be directed from only a limited angular range around of the lead. Segmented electrodes 530, however, can be used to direct stimulus current to a selected angular range around the lead. When segmented electrodes 530 are used in conjunction with an IPG 14 that delivers constant current stimulus, current steering can be achieved to more precisely deliver the stimulus to a position around an axis of the lead (e.g., radial positioning around the axis of the lead). To achieve current steering, segmented electrodes can be utilized in addition to, or as an alternative to, ring electrodes.

The lead 12 includes a lead body 510, terminals 27, and one or more ring electrodes 520 and one or more sets of segmented electrodes 530 (or any other combination of electrodes). The lead body 510 can be formed of a biocompatible, non-conducting material such as, for example, a polymeric material. Suitable polymeric materials include, but are not limited to, silicone, polyurethane, polyurea, polyurethaneurea, polyethylene, or the like. Once implanted in the body, the lead 12 may be in contact with body tissue for extended periods of time. In at least some embodiments, the lead 12 has a cross-sectional diameter of no more than 1.5 millimeters (1.5 mm) and may be in the range of 0.5 to 1.5 mm. In at least some embodiments, the lead 12 has a length of at least 10 centimeters (10 cm) and the length of the lead 12 may be in the range of 10 to 70 cm.

The electrodes 26 can be made using a metal, alloy, conductive oxide, or any other suitable conductive biocompatible material. Examples of suitable materials include, but are not limited to, platinum, platinum iridium alloy, iridium, titanium, tungsten, palladium, palladium rhodium, or the like. Preferably, the electrodes are made of a material that is biocompatible and does not substantially corrode under expected operating conditions in the operating environment for the expected duration of use. Each of the electrodes can either be used or unused (OFF). When the electrode is used, the electrode can be used as an anode or cathode and carry anodic or cathodic current. In some instances, an electrode might be an anode for a period of time and a cathode for a period of time.

Deep brain stimulation leads and other leads may include one or more sets of segmented electrodes. Segmented electrodes may provide for superior current steering than ring electrodes because target structures in deep brain stimulation or other stimulation are not typically symmetric about the axis of the distal electrode array. Instead, a target may be located on one side of a plane running through the axis of the lead. Through the use of a radially segmented electrode array (“RSEA”), current steering can be performed not only along a length of the lead but also around a circumference of the lead. This provides precise three-dimensional targeting and delivery of the current stimulus to neural target tissue, while potentially avoiding stimulation of other tissue.

Any number of segmented electrodes 530 may be disposed on the lead body 510 including, for example, anywhere from one to sixteen or more segmented electrodes 530. It will be understood that any number of segmented electrodes 530 may be disposed along the length of the lead body 510. A segmented electrode 530 typically extends only 75%, 67%, 60%, 50%, 40%, 33%, 25%, 20%, 17%, 15%, or less around the circumference of the lead.

The segmented electrodes 530 may be grouped into sets of segmented electrodes, where each set is disposed around a circumference of the lead 12 at a particular longitudinal portion of the lead 12. The lead 12 may have any number segmented electrodes 530 in a given set of segmented electrodes. The lead 12 may have one, two, three, four, five, six, seven, eight, or more segmented electrodes 530 in a given set. In at least some embodiments, each set of segmented electrodes 530 of the lead 12 contains the same number of segmented electrodes 530. The segmented electrodes 530 disposed on the lead 12 may include a different number of electrodes than at least one other set of segmented electrodes 530 disposed on the lead 12. The segmented electrodes 530 may vary in size and shape. In some embodiments, the segmented electrodes 530 are all of the same size, shape, diameter, width or area or any combination thereof. In some embodiments, the segmented electrodes 530 of each circumferential set (or even all segmented electrodes disposed on the lead 12) may be identical in size and shape.

Each set of segmented electrodes 530 may be disposed around the circumference of the lead body 510 to form a substantially cylindrical shape around the lead body 510. The spacing between individual electrodes of a given set of the segmented electrodes may be the same, or different from, the spacing between individual electrodes of another set of segmented electrodes on the lead 12. In at least some embodiments, equal spaces, gaps or cutouts are disposed between each segmented electrode 530 around the circumference of the lead body 510. In other embodiments, the spaces, gaps or cutouts between the segmented electrodes 530 may differ in size, or cutouts between segmented electrodes 530 may be uniform for a particular set of the segmented electrodes 530 or for all sets of the segmented electrodes 530. The sets of segmented electrodes 530 may be positioned in irregular or regular intervals along a length the lead body 510.

Conductor wires (not shown) that attach to the ring electrodes 520 or segmented electrodes 530 extend along the lead body 510. These conductor wires may extend through the material of the lead 12 or along one or more lumens defined by the lead 12, or both. The conductor wires couple the electrodes 520, 530 to the terminals 27.

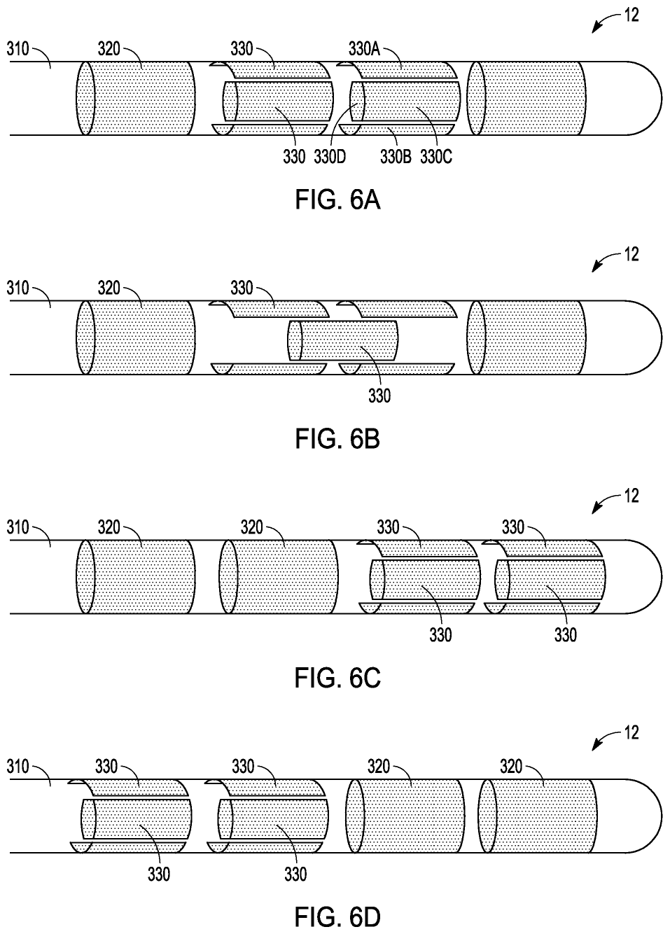

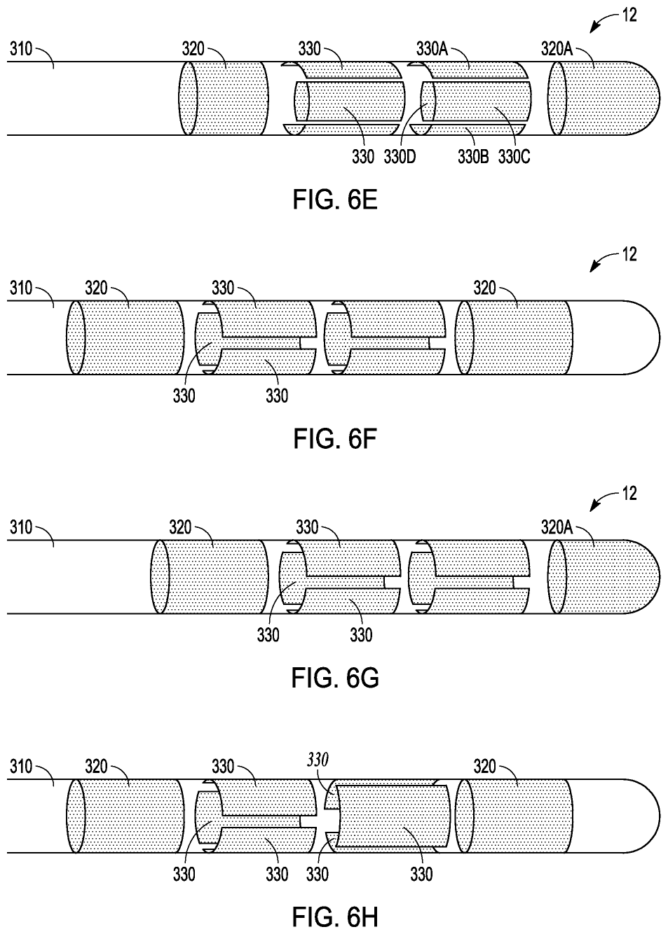

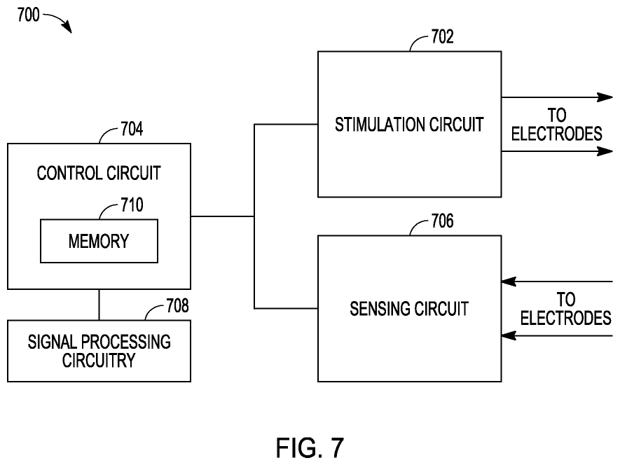

FIGS. 6A-6H are illustrations of different embodiments of leads 12 with segmented electrodes 330, optional ring electrodes 320 or tip electrodes 320a, and a lead body 310. The sets of segmented electrodes 330 each include either two (FIG. 6B), three (FIGS. 6E-6H), or four (FIGS. 6A, 6C, and 6D) or any other number of segmented electrodes including, for example, three, five, six, or more. The sets of segmented electrodes 330 can be aligned with each other (FIGS. 6A-6G) or staggered (FIG. 6H).

When the lead 12 includes both ring electrodes 320 and segmented electrodes 330, the ring electrodes 320 and the segmented electrodes 330 may be arranged in any suitable configuration. For example, when the lead 12 includes two ring electrodes 320 and two sets of segmented electrodes 330, the ring electrodes 120 can flank the two sets of segmented electrodes 330 (see e.g., FIGS. 5, 6A, and 6E-6H, ring electrodes 320 and segmented electrode 330). Alternately, the two sets of ring electrodes 320 can be disposed proximal to the two sets of segmented electrodes 330 (see e.g., FIG. 6C, ring electrodes 320 and segmented electrode 330), or the two sets of ring electrodes 320 can be disposed distal to the two sets of segmented electrodes 330 (see e.g., FIG. 6D, ring electrodes 320 and segmented electrode 330). One of the ring electrodes can be a tip electrode (see e.g., tip electrode 320a of FIGS. 36E and 6G). It will be understood that other configurations are possible as well (e.g., alternating ring and segmented electrodes, or the like).

By varying the location of the segmented electrodes 330, different coverage of the target neurons may be selected. For example, the electrode arrangement of FIG. 6C may be useful if the physician anticipates that the neural target will be closer to a distal tip of the lead body 310, while the electrode arrangement of FIG. 6D may be useful if the physician anticipates that the neural target will be closer to a proximal end of the lead body 310.

Any combination of ring electrodes 320 and segmented electrodes 330 may be disposed on the lead 12. For example, the lead 12 may include a first ring electrode 320, two sets of segmented electrodes; each set formed of four segmented electrodes 330, and a final ring electrode 320 at the end of the lead. This configuration may simply be referred to as a 1-4-4-1 configuration (FIGS. 6A and 6E, ring electrodes 320 and segmented electrode 330). It may be useful to refer to the electrodes with this shorthand notation. Thus, the embodiment of FIG. 6C may be referred to as a 1-1-4-4 configuration, while the embodiment of FIG. 6D may be referred to as a 4-4-1-1 configuration. The embodiments of FIGS. 6F, 6G, and 6H can be referred to as a 1-3-3-1 configuration. Other electrode configurations include, for example, a 2-2-2-2 configuration, where four sets of segmented electrodes are disposed on the lead, and a 4-4 configuration, where two sets of segmented electrodes, each having four segmented electrodes 330 are disposed on the lead. The 1-3-3-1 electrode configuration of FIGS. 6F, 6G, and 6H has two sets of segmented electrodes, each set containing three electrodes disposed around the circumference of the lead, flanked by two ring electrodes (FIGS. 6F and 6H) or a ring electrode and a tip electrode (FIG. 6G). In some embodiments, the lead includes 16 electrodes. Possible configurations for a 16-electrode lead include but are not limited to 4-4-4-4; 8-8; 3-3-3-3-3-1 (and all rearrangements of this configuration); and 2-2-2-2-2-2-2-2.

Any other suitable arrangements of segmented and/or ring electrodes can be used. As an example, arrangements in which segmented electrodes are arranged helically with respect to each other. One embodiment includes a double helix. One or more electrical stimulation leads can be implanted in the body of a patient (for example, in the brain or spinal cord of the patient) and used to stimulate surrounding tissue. The lead(s) are coupled to the implantable pulse generator (such as IPG 14 in FIG. 2).

FIG. 7 is a block diagram of portions of an embodiment of a neurostimulation system 700 for providing neurostimulation. The neurostimulation system 700 includes a stimulation circuit 702, a control circuit 704, and a sensing circuit 706. The stimulation circuit 702 can be operatively coupled to stimulation electrodes such as any of the electrodes described herein and the stimulation circuit 702 provides or delivers electrical neurostimulation energy to the electrodes. The control circuit 704 can include a processor such as a microprocessor, a digital signal processor, application specific integrated circuit (ASIC), or other type of processor, interpreting or executing instructions in software modules or firmware modules. The instructions can be stored in memory 710 that can be integral to the control circuit 704 or separate from the control circuit 704. The control circuit 704 can include other circuits or sub-circuits to perform the functions described. These circuits may include software, hardware, firmware, or any combination thereof. Multiple functions can be performed in one or more of the circuits or sub-circuits as desired.

The neurostimulation system 700 includes a sensing circuit 706. An example of the sensing circuit 706 includes one or more sense amplifiers switchable among recording electrodes to sense internal neural signals of the patient. The neurostimulation system 700 includes signal processing circuitry 708. The signal processing circuitry 708 can include one or more processes running on a processor to perform signal analysis or other signal processing on the neural signals sensed using the sensing circuit 706.

The neurostimulation system 700 is connected to at least one stimulation lead (e.g., stimulation lead 12 in FIG. 5) that can be implanted in the body of a patient (for example, in the brain or spinal cord of the patient) and the electrodes of the stimulation leads are used to stimulate surrounding tissue. The neurostimulation system 700 can be used to evaluate the placement of the stimulation lead. In some examples, the neurostimulation system 700 is used at the time of implanting to evaluate the placement of the stimulation lead. The stimulation circuit 702, sensing circuit 706, control circuit 704, and signal processing circuitry 708 can be included in the same device of the system to evaluate the lead position. In some examples, the neurostimulation system 700 is used some time after implant to evaluate the efficacy of the electrode configuration, or aid in producing efficacious neurostimulator settings. The stimulation circuit 702, sensing circuit 706, and control circuit 704 can be included in an IPG (e.g., IPG 14 in FIG. 2) and the signal processing circuitry 708 can be included in a programming device (e.g., CP 18 in FIG. 2).

When appropriately placed, the stimulation lead 12 provides stimulation energy (e.g., electrical current) to the tissue of the neurostimulation target and the stimulation produces the intended benefit to the patient with a minimum of side effects. Proper lead placement may also lead to lower energy needed to produce the desired result. When the lead is not properly placed, the neurostimulation may result in one or both of lack of therapy and unintended side effects.

The human nervous system produces a neural response to neurostimulation received via sensory receptors or received directly into any part of the network of neural elements that forms the nervous system. Additionally, neurostimulation can excite elements of the human nervous system in a manner that produces neural responses. These neural responses are known as evoked potentials. Evoked potential (EP) signals can be sensed electrically by the neurostimulation system 700, such as by using sense amplifiers of the sensing circuit 706 coupled to recording electrodes for example. A repetitive stimulus can be applied or presented to the nervous system and the evoked potential signals (e.g., evoked resonant neural activity (ERNA) signals) sensed from the presentations can be processed (e.g., filtered by averaging) to detect presence of evoked potentials.

Changes in the sensed evoked potential signals can provide information regarding the efficacy of placement of the stimulation lead. The way in which the evoked potential signals change in response to one or more of changes in the number of presentations of the stimulation, changes in timing or conditions of the stimulation, and changes in the properties of the stimulation can provide information of the proximity (e.g., distance and direction) of the stimulation lead to the preferred lead location for the neurostimulation. The preferred lead location for the neurostimulation may be a target tissue volume, or sub-volume, a preferred stimulation location, a stimulation location where evoking stimulation energy affect electrophysiology (EP) responses in the desired manner (e.g., by maximizing a particular EP response feature), or a location where stimulation produces the desired response (e.g., maximizing a therapeutic effect, counteracting or minimizing a side effect, etc.).

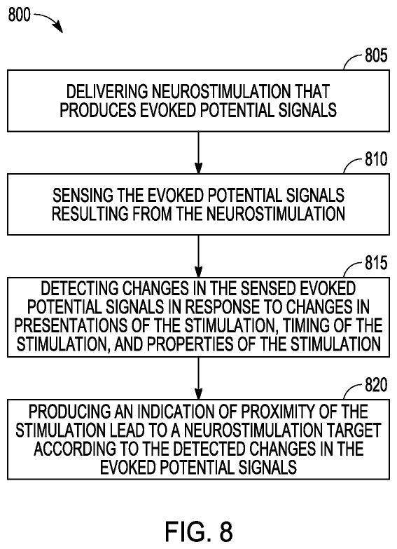

FIG. 8 is a flow diagram of an example of operating a neurostimulation system to deliver electrical neurostimulation. The system includes a stimulation circuit (e.g., stimulation circuit 702 in FIG. 7) operatively coupled to a stimulation lead (e.g., stimulation lead 12 in FIG. 2) that is implanted in a patient or subject. The stimulation lead can include multiple electrodes, including one or both of ring electrode and segmented electrodes. At block 805, neurostimulation is delivered to the patient using the stimulation lead. The neurostimulation produces an evoked potential signal in the patient's nervous system. At block 810, the evoked potential signals resulting from the neurostimulation are sensed and can be recorded by being stored in memory of the system. The sensed evoked potential signals are used to determine if the stimulation lead is in the desired position.

At block 815, the sensed evoked potential signals are processed (e.g., by the signal processing circuitry 708 in FIG. 7), and changes in the evoked potential signals are detected. The changes can be used by the system to determine the proximity of the lead to the target, such as whether the lead is on the target, off of the target, or far from the target. The changes that provide the proximity information can be one or more of changes that occur in the evoked potential signals across multiple presentations of the neurostimulation, changes in the evoked potential signals that occur across changes in timing of the neurostimulation, and changes in the evoked potential signals across changes in parameters of the neurostimulation. At block 820, the system can produce an indication of the proximity of the lead and present the indication to the user or provide the indication to a separate device. The proximity information can be helpful in deciding whether the lead placement is appropriate or can be improved.

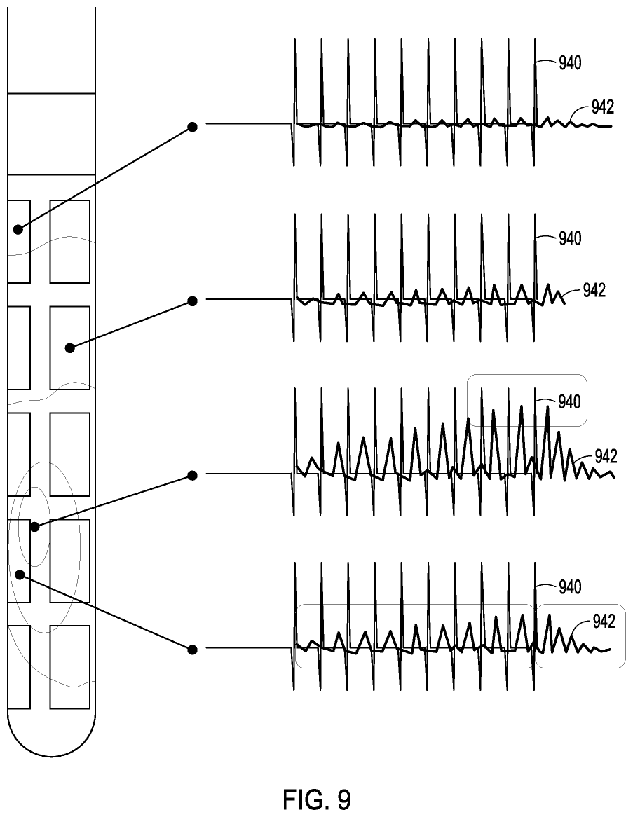

FIG. 9 is an illustration of the variation in a sensed neural response with the distribution of the electrodes at different levels of the stimulation lead 12. The stimulation lead includes 5 levels of segmented electrodes. FIG. 9 includes a simulation artifact 940 that is a burst of pulses (e.g., 10 pulses) delivered using the electrodes. The pulses of the bursts may have the same amplitude and pulse width, or varying amplitude and pulse width. The electrodes are used to sense evoked potential signals produced by the stimulation. FIG. 9 also shows evoked potential signal waveforms 942 sensed at the electrode levels. FIG. 9 shows that the amplitude of the evoked potential signals changes with the level of the electrodes.

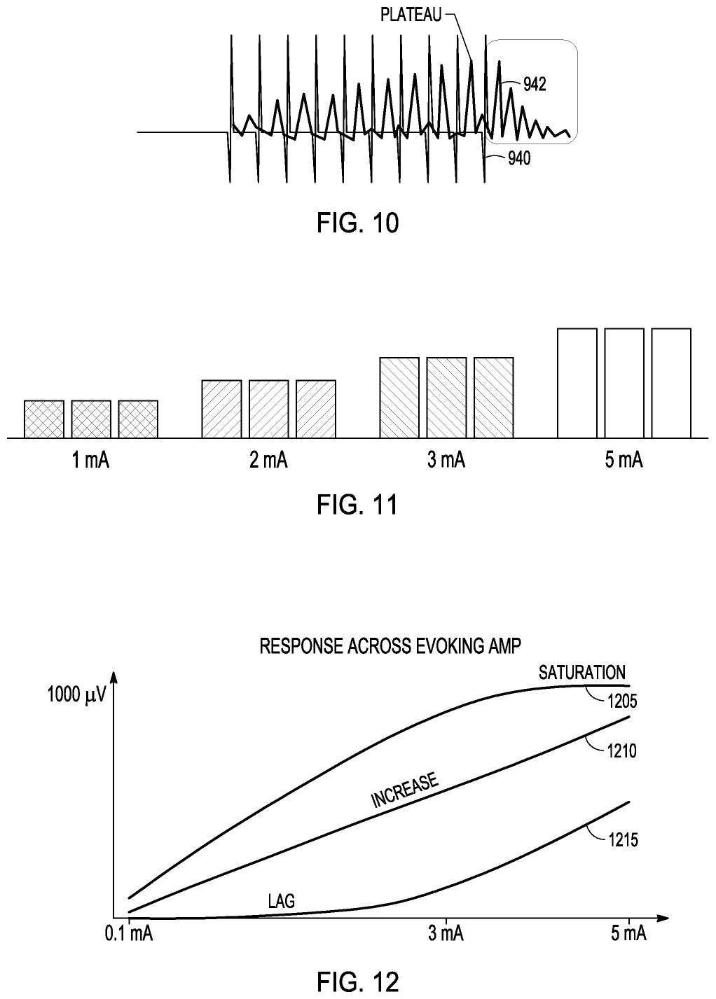

FIG. 10 is an illustration of the stimulation artifact 940 of FIG. 9 and the evoked potential signal 942 having the largest amplitude. The stimulation artifact 940 includes a burst of pulses and the evoked potential signal 942 includes peaks between pulses of the burst. The amplitude of the peaks increases with each pulse of the burst until near the end of the burst, where the amplitude plateaus and doesn't increase. The plateau indicates a saturation of the neural response during the burst. Saturation of the neural response is where a feature of the neural response approaches and reaches a maximum or minimum value.

The saturation evident in the evoked potential signal provides proximity information and may indicate that the stimulation lead is very near or on the neurostimulation target. The neurostimulation system may apply a burst of stimulation pulses and sense the evoked response signal at one or more electrodes of the stimulation lead. The signal processing circuitry of the neurostimulation system may produce an indication of the proximity of the stimulation lead to the target when detecting saturation in the sensed evoked potential signal. For example, the user interface of the neurostimulation system may include a display, and the system displays whether the stimulation is very near the target or away from the target according to whether the system detects saturation in the sensed evoked potential signals.

According to some examples, the neurostimulation system may present a repetitive stimulus to the target tissue while changing a parameter of the stimulus. The system determines proximity information from the changes in the sensed evoked potential signals produced by the stimuli. FIG. 11 is an illustration of delivering bursts of pulses to the target, while changing the amplitude of the pulses. In variations, the pulse width of the pulses may change, and the amplitude may be the same. The neurostimulation system measures changes in the evoked potential signal across the changes in amplitude. In the example of FIG. 11, the amplitude of the bursts is changed from 1 milliamp (1 mA) to 5 mA. The bursts in FIG. 11 include 3 pulses for simplicity of the drawing, but the bursts may include more pulses.

FIG. 12 is an illustration of the results of delivering neurostimulation pulses across increasing amplitude for three different simulation lead placements. The horizontal axis is the amplitude of the stimulation in mA and the vertical axis is the amplitude of the evoked response signals in microvolts (μV). In plot 1205, the sensed evoked response signals show saturation. The neurostimulation system may provide an indication to the user that the stimulation lead is close enough to the target to cause saturation in the response to the stimulation. This may lead the user to conclude that the placement of the lead is appropriate.

In plot 1210, the sensed evoked response signals show an increase in amplitude proportional to the increase in amplitude of the stimulation but does not show saturation. Instead, the amplitude of the sensed evoked response signals increases monotonically with the increase in amplitude of the stimulation. The neurostimulation system may provide an indication to the user that the stimulation lead is close enough to the target to increase recruitment in the evoked potential signals, but not close enough to cause saturation in the response to the stimulation. This result may lead the user to conclude that the placement of the lead is appropriate, or the user may want to reposition the stimulation lead.

In plot 1215, the sensed evoked response signals show a lag in the increase in amplitude compared to the increase in amplitude of the stimulation. The plot 1215 shows that the evoked response signals increase slowly in amplitude at a first rate until the stimulation amplitude nears a threshold near 3 mA, and then increases at a different faster rate. The neurostimulation system may provide an indication to the user that the stimulation lead is far enough away from the target that increasing the stimulation amplitude does not lead to a significant increase in the response to the stimulation. This result may lead the user to conclude that the stimulation lead needs to be repositioned. In some examples, the neurostimulation system only presents the proximity information to the user, and in certain examples the neurostimulation system may provide a recommendation on transitioning the lead to another position or another electrode configuration based on the results of the measurements.

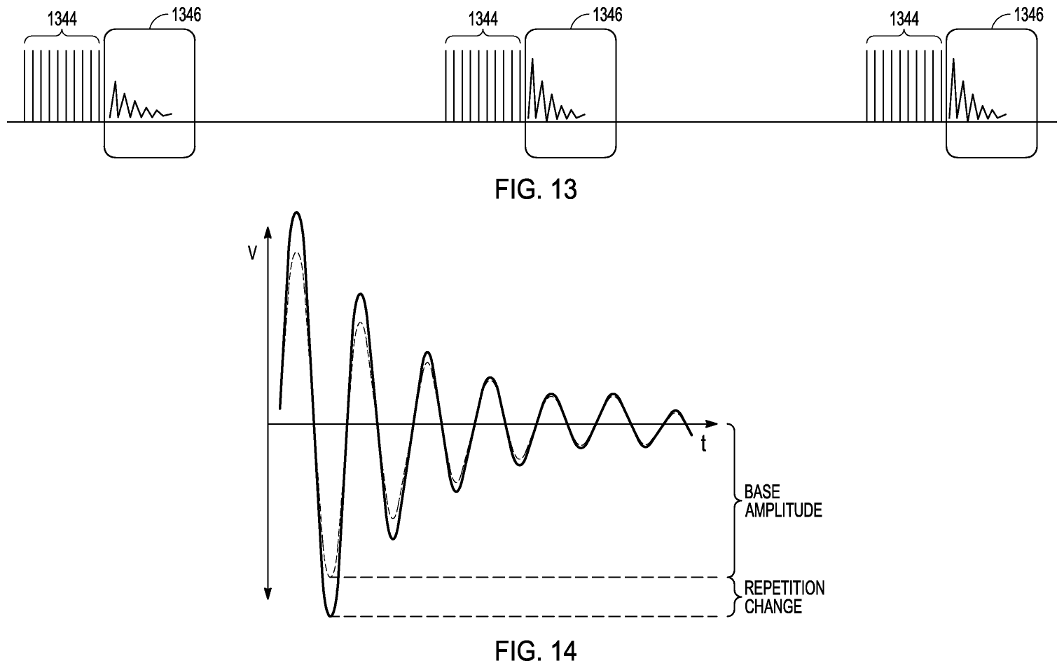

The neurostimulation system may determine proximity information using other changes in the evoked response signals. In some examples, the neurostimulation system looks for changes across multiple presentations of the neurostimulation. FIG. 13 is an illustration of waveforms of multiple presentations of a burst of neurostimulation pulses 1344 and the evoked response signals resulting from the multiple bursts of neurostimulation pulses 1344. The neurostimulation system looks for changes in the evoked response signals between the bursts of pulses. In the example of FIG. 13, the neurostimulation system measures the amplitude of the peak of the sensed evoked potential signals 1346 after the last pulse at the end of the bursts of pulses (e.g., the first positive peak of the signal or P1). The changes in the peaks can provide proximity information. In FIG. 13, the post-burst evoked potential signals have a peak amplitude immediately following the last pulse of the burst. The peak amplitude saturates over the multiple presentations of the bursts.

FIG. 14 is another illustration of waveforms of post-burst evoked potential signals. Two waveforms are superimposed over each other and show an increase in the amplitude of the negative first peak (N1 peak) from a base amplitude to a larger amplitude with repetition of the bursts. The signal processing circuitry of the neurostimulation system may produce an indication of the proximity of the stimulation lead to the target according to whether the peaks show saturation, a significant increase in amplitude but not saturation, or a slow change or no change in amplitude.

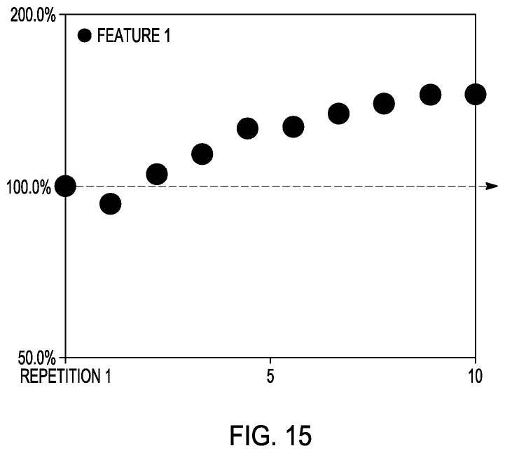

FIG. 15 is a graph showing percentage change in magnitude of a feature of a sensed evoked signal (e.g., P1 peak) as a function of the number of presentations or repetitions of the evoking neurostimulation. The graph shows saturation of the sensed evoked response signal after about 8 presentations of the evoking neurostimulation. The signal processing circuitry of the neurostimulation system may produce an indication of the proximity of the stimulation lead to the target, may present settings recommended for therapeutic stimulation, or may modify algorithms used to determine proximity and settings, based on the saturation in the evoked response signal.

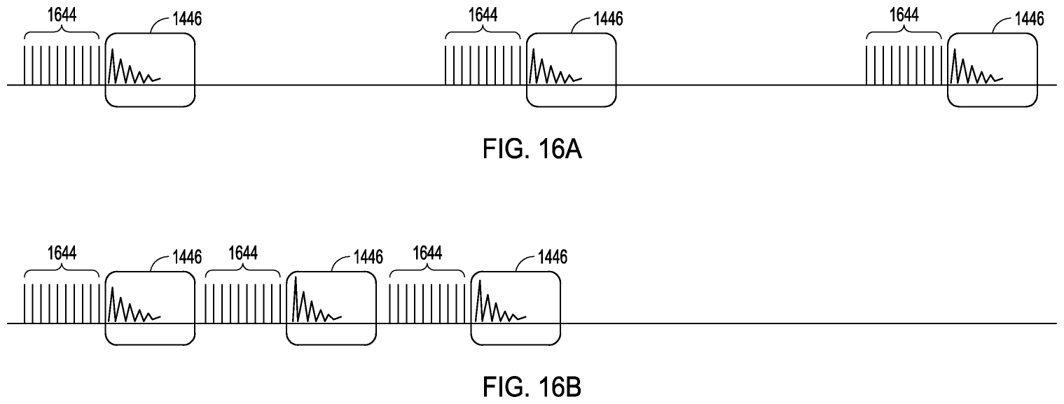

In some examples, changes in the repeating evoking neurostimulation can provide proximity information. FIG. 16A is an illustration of waveforms of repetitions of a burst of neurostimulation pulses 1644 and the sensed 1646 evoked response signals resulting from the repeating bursts of neurostimulation pulses 1644. FIG. 16B shows a change in subsequent change in the repeating bursts. The time between bursts of neurostimulation pulses 1644 (inter-burst time) is decreased.

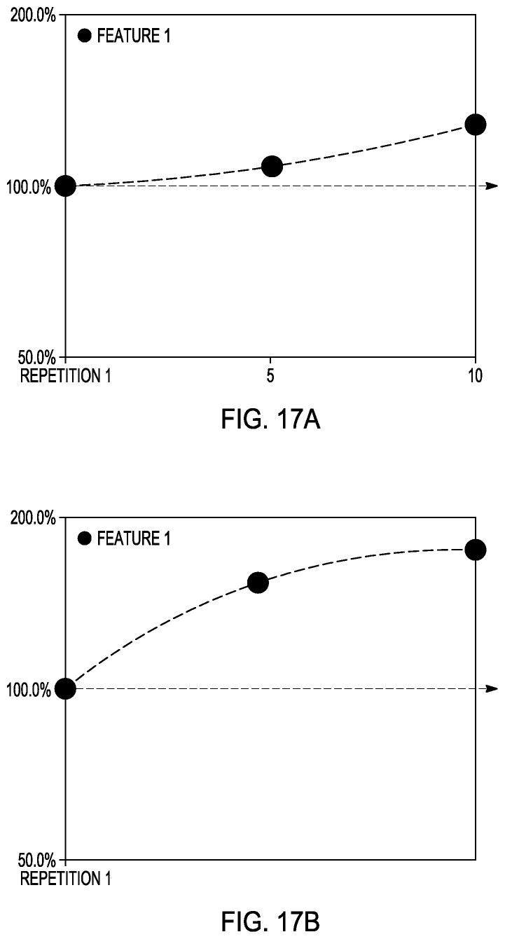

FIG. 17A is a graph showing the change in magnitude of a feature of a sensed evoked signal resulting from the presentations of neurostimulation of FIG. 16A, and FIG. 17B is a graph showing the change in magnitude of the feature of a sensed evoked signal resulting from the presentations of neurostimulation of FIG. 16B. FIGS. 17A and 17B show that the presentations in FIG. 16B result in saturation while the presentations of FIG. 16A do not show saturation. The changes in evoked response signals due to the changes between the neurostimulation can provide proximity information.

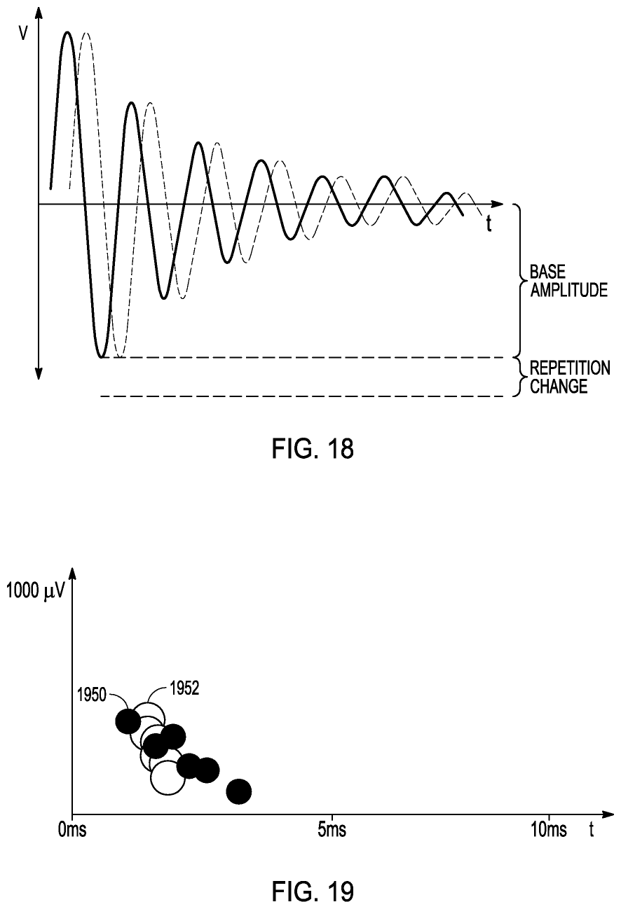

Changes in parameters other than amplitude or magnitude over time may provide proximity information for the stimulation lead. FIG. 18 is another illustration of waveforms of post-burst evoked potential signals. Again, two waveforms are superimposed. The waveforms show a time delay or phase change between the earlier base evoked potential signal and a subsequent evoked potential signal. The time delay may provide proximity information regarding the stimulation lead.

FIG. 19 is a plot of small neural responses 1950 and large neural responses 1952. Small responses that occur earlier in time after the stimulation may indicate a closer lead position to the target than responses that are larger in amplitude and occur later in time.

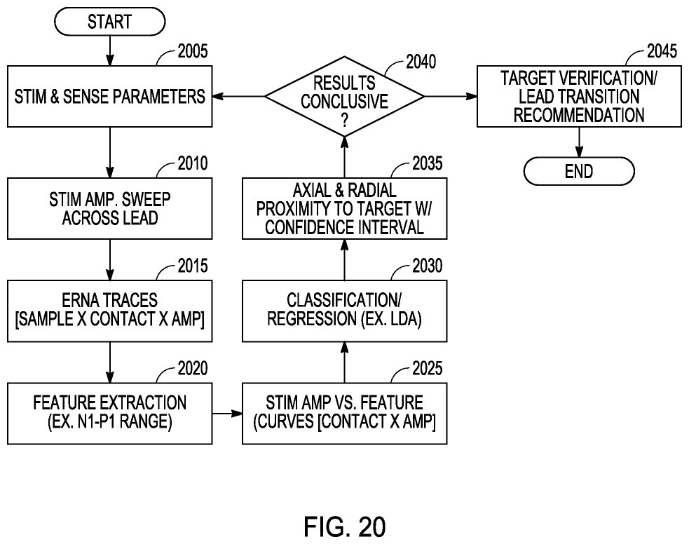

FIG. 20 is a flow diagram of an example of evaluating the position of a stimulation lead when the lead is connected to a neurostimulation system. Stimulation amplitude is changed and the changes in sensed evoked potential signals resulting from the stimulation are determined. At block 2005, parameters of the neurostimulation parameters and sensing parameters are set in the neurostimulation device. The parameters may be set by the user, or the parameters may be programmed into the neurostimulation system.

At block 2010, the neurostimulation system performs one or more sweeps of stimulation amplitude across the electrodes of the leads. Each sweep may produce a set of evoked potential signals (e.g., ERNA traces) as in the example of FIG. 9 for a different amplitude. At block 2015, the ERNA traces may be recorded according to sample, electrode or contact of the lead, and amplitude.

At block 2020, features of the evoked potential signals are extracted from the sensed signals. For instance, the amplitude of one or both of the N1 peak and the P1 peak may be extracted by signal processing circuitry of the system. In certain examples, the timing of the peaks is extracted. At block 2025, the neurostimulation system produces data related to changes in the extracted features that occur with changes in stimulation amplitude. At block 2030, the neurostimulation system may perform a regression analysis (e.g., linear discriminant analysis (LDA)) to identify a relationship of the extracted features and the stimulation amplitude.

At block 2035, information related to the proximity of the stimulation lead to the neurostimulation target is determined using the data. The proximity information includes radial proximity information and whether the lead is near the target or off of the target. In some examples, the proximity information includes axial proximity to the target. The evoked potential signals from different levels of electrodes can be compared to determine axial proximity of the stimulation lead to the target. A confidence interval can be determined for the proximity information based on the measurements.

At block 2040, it is determined if the results are conclusive. In some examples, at block 2045 the neurostimulation system provides a recommendation based on the measurements regarding one or both of the position of the lead and the electrode configuration. In variations, the neurostimulation may transition the lead to another electrode configuration autonomously. In some examples, raw data is provided to the user and the user decides whether the data indicates that the desired stimulation of the target is verified or if the data indicates the stimulation lead configuration should be transitioned to change position or electrode configuration. Optionally, enhanced data may be produced, and the enhanced data is presented to the user for use in deciding whether to transition the lead. The neurostimulation system may present enhanced data to the user with the user interface.

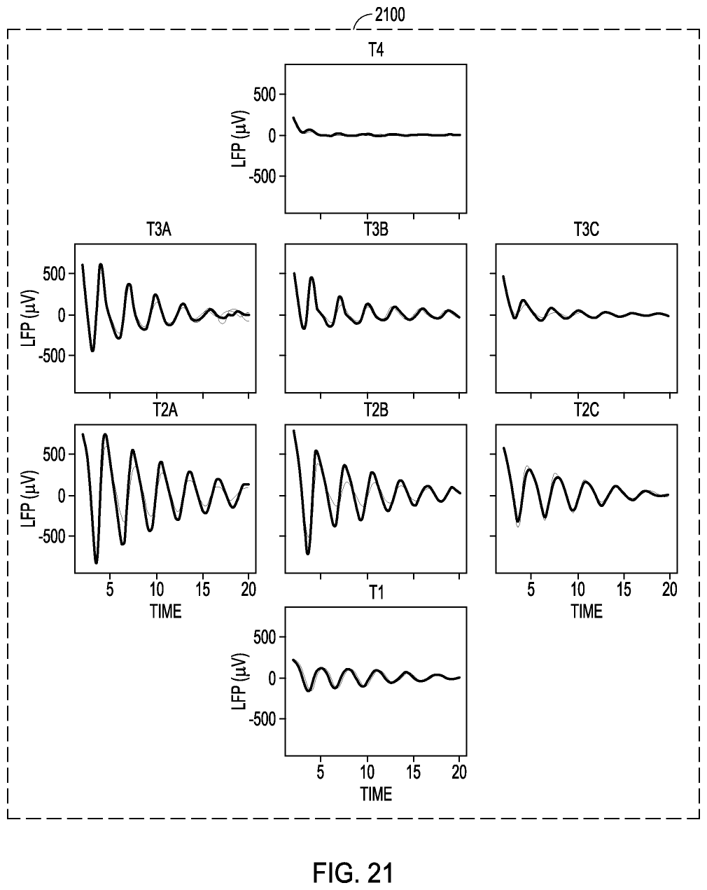

FIG. 21 is an illustration of an example of a display 2100 with enhanced data that can be presented on a user interface of a neurostimulation system. The display 2100 represents a spatial view of the electrodes of the stimulation lead. The top and bottom squares represent top and bottom ring electrodes of the stimulation lead. The six middles squares represent segmented electrodes of the stimulation lead. Each segmented electrode is comprised of three segments. The display 2100 can be used to show the sensed evoked response signals resulting from the stimulation applied using the lead. The results are presented simultaneously in the display. Ten repetitions of neurostimulation were applied to the target using the stimulation lead and ERNA signals were sensed using the electrodes. The sensed ERNA signals are shown super imposed in the squares. The display 2100 shows how the root mean square amplitude of the signal changed at the electrodes during the repetitions of the stimulation. Showing the results for each electrode shows the difference in the sensed ERNA signals across the space of the electrodes of the stimulation lead.

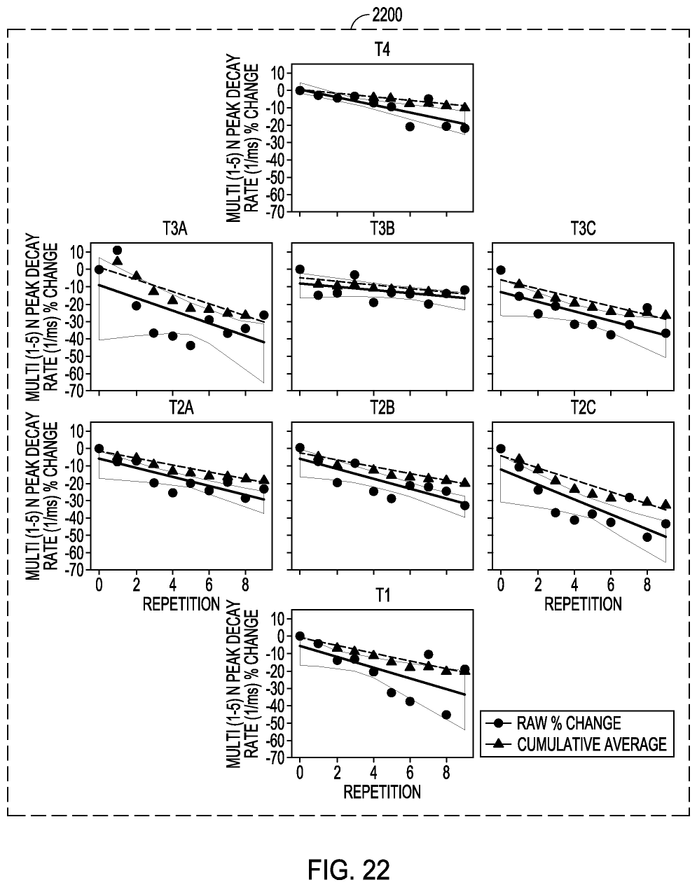

FIG. 22 is an illustration of another example of a display 220 with enhanced data that can be presented on the user interface of a neurostimulation system. As in the example of FIG. 21, the display 220 represents a spatial view of the electrodes of the stimulation lead. The example of FIG. 22 displays the rate of decay of the negative peak (N peak) of the sensed signals as a function of the presentation of the neurostimulation. The spatial aspect of the information may help the user determine whether to reposition the stimulation lead, or to select electrodes to use for the neurostimulation, such as by choosing electrodes that show a slower or lesser decay in the sensed evoked response signal.

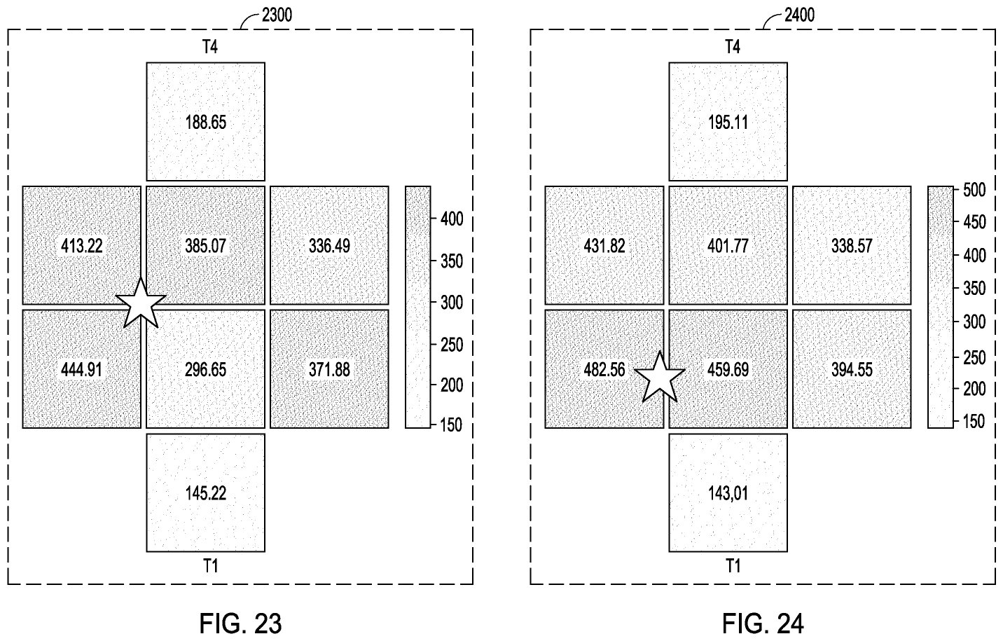

FIGS. 23 and 24 illustrate additional examples of displays 2300, 2400 that can be presented on a user interface of a neurostimulation system. The examples present the time series data of FIG. 21 in a different way. Display 2300 shows only the result of the first application of the ten repetitions of the neurostimulation in FIG. 21. The value of the RMS peak is given a score and the shading of the display 2300 reflects the score. Display 2400 shows only the result of the tenth application of the neurostimulation. The stars in the displays show the movement of the center of the distribution between the first and the tenth application of the stimulation. The user may make different decisions based on the size and movement of the peak shown in the displays 2300, 2400. If the peak in the ERNA signal has a large amplitude or magnitude and did not move much over the ten repetitions of stimulation, this may indicate a hot spot of the neural response. The user may make a different decision about the stimulation this result than if the peak in the ERNA signal has a large amplitude and moved a lot or if the peak in the ERNA signal has a small amplitude and moved a lot.

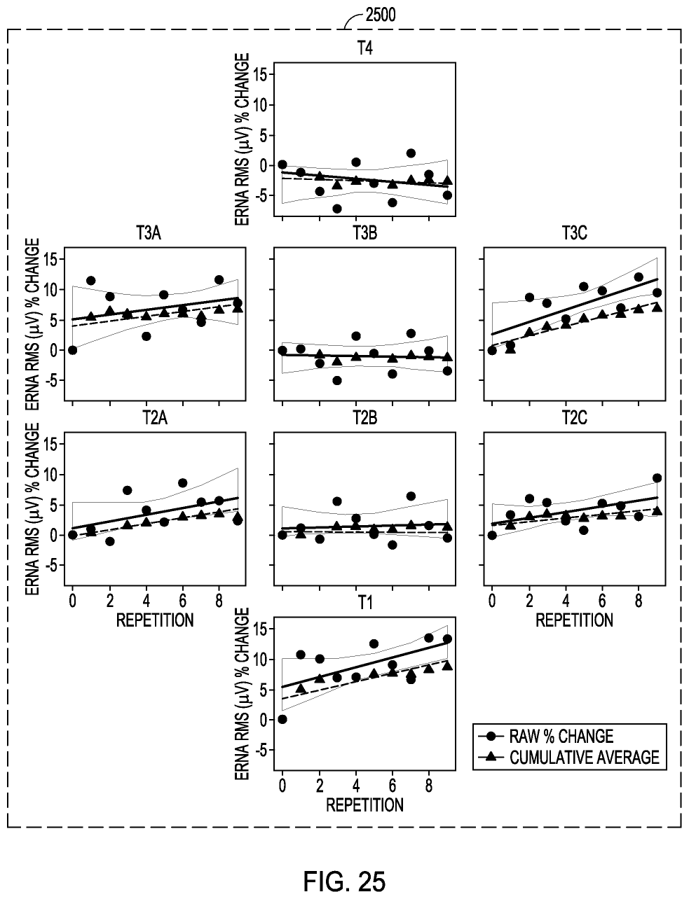

FIG. 25 is an illustration of another example of a display 2500 showing enhanced data that can be presented on a user interface of a neurostimulation system. The data in the display may be derived from the data in the display example of FIG. 21. Each square of the display shows a graph of the ERNA RMS peak sensed with respect to that electrode or electrode segment versus the repetition number of the stimulation. The line in the graphs shows a regression for the data, and the shaded region in the graphs shows a confidence level for the data. A flat response with a large amplitude tells the user that they are getting a stronger ERNA response for these areas. If the user wants a larger ERNA response in that area, the graph tells the user that this is a good lead placement. A sloped response starting from a small amplitude tells the user that the ERNA response is small and is growing with the repeated applications of stimulation. This provides two pieces of information that can be useful to the user. When a user sees that a response is growing across presentations, they may choose to alter the evoking parameters to search for responses which saturate, and use those saturated responses in decision making

In some examples, the neurostimulation system makes a recommendation to the user based on the data. For example, the system may highlight an ERNA response a large amplitude and flat response to the user and recommend using the current lead location and electrode configuration. In another example, the system may recommend changing the lead position based on a flat response with a small amplitude due to a poor ERNA response. In a further example, the user might compare responses with respect to two electrodes and see in the first a response which is flat and in the second a response which is growing. In the case that the magnitudes of the responses were similar for the last presentation recorded, the user may conclude that the response was likely to further increase for the second electrode and use that information in further decision making. Slope lines can be fit to response data to aid in this process.

Stimulation lead placement for neurostimulation can be complicated and time consuming. The several embodiments described herein provide device-based assistance in lead placement. Evoking neural responses and measuring changes in the responses can provide information useful in guiding lead placement.

The embodiments described herein can be methods that are machine or computer-implemented at least in part. Some embodiments may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. Various embodiments are illustrated in the figures above. One or more features from one or more of these embodiments may be combined to form other embodiments.

The above detailed description is intended to be illustrative, and not restrictive. The scope of the disclosure should, therefore, be determined with references to the appended claims, along with the full scope of equivalents to which such claims are entitled.

What is claimed is:

1. A computer-implemented method of operating a neurostimulation device to deliver electrical neurostimulation when connected to an implantable stimulation lead, the method comprising:
delivering first neurostimulation to a subject that produces evoked potential signals;
delivering second neurostimulation that includes at least one of multiple presentations of the first neurostimulation or a change in a parameter of the first neurostimulation;
sensing the evoked potential signals resulting from the first neurostimulation and the second neurostimulation;
detecting changes in the sensed evoked potential signals between the first neurostimulation and the second neurostimulation; and
producing an indication of proximity of the stimulation lead to a preferred lead location according to the detected changes in the sensed evoked potential signals.

2. The method of claim 1,
wherein the delivering the second neurostimulation includes delivering multiple neurostimulation pulses;
wherein the detecting changes in the sensed evoked potential signals includes detecting a change in a signal feature of the sensed evoked potential signals between neurostimulation pulses; and
wherein the producing the indication of proximity of the stimulation lead includes determining proximity of the stimulation lead according to a degree of saturation in the sensed evoked potential signals.

3. The method of claim 1,
wherein the delivering the second neurostimulation includes changing an amplitude of the neurostimulation;
wherein the detecting changes in the sensed evoked potential signals includes detecting a change in one or more features of the sensed evoked potential signals; and
wherein the producing an indication of proximity of the stimulation lead includes determining proximity of the stimulation lead according to a degree of saturation in the sensed evoked potential signals.

4. The method of claim 1,
wherein the delivering the second neurostimulation includes delivering multiple bursts of neurostimulation pulses;
wherein the sensing the evoked potential signals includes sensing a post-burst evoked potential signal after a last pulse of a burst of neurostimulation pulses; and
wherein the detecting changes in the sensed evoked potential signals includes detecting a change in magnitude of a feature of the post-burst evoked potential signal over the multiple bursts; and
wherein producing the indication of proximity of the stimulation lead includes determining proximity of the stimulation lead according to the detected change in magnitude of the post-burst evoked potential signal.

5. The method of claim 1,
wherein the delivering the second neurostimulation includes delivering multiple bursts of neurostimulation pulses;
wherein the sensing the evoked potential signals includes sensing a post-burst evoked potential signal after a burst of neurostimulation pulses;
wherein the detecting changes in the sensed evoked potential signals includes detecting a phase change of the post-burst evoked potential signal; and
wherein producing the indication of proximity of the stimulation lead includes producing the indication according to the detected phase change.

6. The method of claim 1,
wherein the delivering the second neurostimulation includes delivering multiple bursts of neurostimulation pulses;
wherein the sensing the evoked potential signals includes sensing a post-burst evoked potential signal after a last pulse of a burst of neurostimulation pulses;
wherein the detecting changes in the sensed evoked potential signals includes detecting changes in timing of a feature of the post-burst evoked potential signal over the multiple bursts; and
wherein producing the indication of proximity of the stimulation lead includes producing the indication according to the detected changes in timing of the feature of the post-burst evoked potential signal.

7. The method of claim 1, wherein producing the indication of proximity of the stimulation lead includes:
presenting an indication that the stimulation lead is close to the neurostimulation target according to a degree of saturation in the sensed evoked potential signals;
presenting an indication that the stimulation lead is off of the preferred lead location according to a monotonical increase in amplitude of the sensed evoked potential signals; and
presenting an indication that the stimulation lead is not close to the preferred lead location according to an amplitude of the sensed evoked potential signals that increases at first slower rate and then increases at second faster rate.

8. The method of claim 1, wherein producing the indication of proximity of the stimulation lead includes:
presenting an indication that the stimulation lead is close to the preferred lead location when the sensed evoked potential signals saturate at a first signal amplitude at a first time relative to the neurostimulation; and
presenting an indication that the stimulation lead is not close to the preferred lead location when the sensed evoked potential signals saturate at a second signal amplitude at a second time relative to the neurostimulation, wherein the second signal amplitude is larger than the first signal amplitude and the second time is later than the first time relative to the neurostimulation.

9. A neurostimulation system, the system comprising:
a stimulation circuit configured to deliver electrical neurostimulation to a subject when coupled to an implantable stimulation lead;
a sensing circuit configured to sense evoked potential signals when coupled to the stimulation lead;
a control circuit operatively coupled to the stimulation circuit and the sensing circuit, and configured to initiate delivery of neurostimulation to the subject that produces an evoked potential signal, and record sensed evoked potential signals resulting from the neurostimulation; and
signal processing circuitry configured to detect changes in the sensed evoked potential signals in response to a change in at least one of the number of repeated presentations of the neurostimulation or a change in a parameter of the first neurostimulation, and produce an indication of proximity of the stimulation lead to a preferred lead location according to the detected changes in the evoked potential signals.

10. The system of claim 9,
wherein the control circuit is configured to initiate delivery of multiple neurostimulation pulses by the stimulation circuit;
wherein the signal processing circuit is configured to:
detect a change in a signal feature of the sensed evoked potential signals between neurostimulation pulses; and
produce the indication of proximity of the stimulation lead according to a degree of saturation in the sensed evoked potential signals.

11. The system of claim 9,
wherein the control circuit is configured to initiate delivery of multiple neurostimulation pulses by the stimulation circuit, and change an amplitude of the neurostimulation pulses;
wherein the signal processing circuit is configured to:
measure amplitude of the sensed evoked potential signals with the changing amplitude of the neurostimulation; and
produce the indication of proximity of the stimulation lead according to whether the sensed evoked potential signals show saturation.

12. The system of claim 9,
wherein the control circuit is configured to:
initiate delivery of multiple bursts of neurostimulation pulses; and
record a sensed a post-burst evoked potential signal after a last pulse of a burst of neurostimulation pulses; and
wherein the signal processing circuit is configured to:
detect a change in magnitude of a feature of the post-burst evoked potential signal over the multiple bursts; and
produce the indication of proximity of the stimulation lead according to the detected change in magnitude of the post-burst evoked potential signal.

13. The system of claim 9,
wherein the control circuit is configured to:
initiate delivery of multiple bursts of neurostimulation pulses; and
record a post-burst evoked potential signal sensed after at least a last pulse of a burst of neurostimulation pulses; and
wherein the signal processing circuit is configured to:
measure frequency of the post-burst evoked potential signal over the multiple bursts; and
produce the indication of proximity of the stimulation lead according to detected changes in frequency of the post-burst evoked potential signal.

14. The system of claim 9,
wherein the control circuit is configured to:
initiate delivery of multiple bursts of neurostimulation pulses; and
record a sensed a post-burst evoked potential signal after at least a last pulse of a burst of neurostimulation pulses; and
wherein the signal processing circuit is configured to:
detect a feature in the post-burst evoked potential signal;
detect a change in timing of the detected feature over the multiple bursts of neurostimulation pulses; and
produce the indication of proximity of the stimulation lead according to detected changes in timing of the feature of the post-burst evoked potential signal.

15. The system of claim 9, including:
a user interface operatively coupled to the signal processing circuit, and wherein the signal processing circuit is configured to:
detect saturation of a feature in a sensed evoked potential signal and present an indication on the user interface that the stimulation lead is close to the preferred lead location according to a degree of saturation detected in the sensed evoked potential signals;
detect when a magnitude of the sensed evoked potential signal increases monotonically with the neurostimulation and present an indication on the user interface that the stimulation lead is off the preferred lead location when detecting the monotonical increase in the sensed evoked potential signal; and
detect when a rate of increase in the magnitude of the feature in the sensed evoked potential signal changes and present an indication on the user interface that the stimulation lead is not close to the preferred lead location according to the detected change in rate of increase in the magnitude of the feature.

16. The system of claim 9, including:
a user interface operatively coupled to the signal processing circuit, and wherein the signal processing circuit is configured to:
detect when a sensed evoked potential signal saturates at a first signal amplitude at a first time relative to delivery of the neurostimulation and present an indication on the user interface that the stimulation lead is close to the preferred lead location in response to the detection of the first signal amplitude at the first time; and
detect when a sensed evoked potential signal saturates at a second signal amplitude larger than the first signal amplitude at a second time relative to the neurostimulation later than the first time and present an indication on the user interface that the stimulation lead is not close to the preferred lead location in response to the detection of the second signal amplitude at the second time.

17. The system of claim 9, including:
a communication circuit configured to communicate information with a separate device; and
wherein the stimulation circuit, sensing circuit, and communication circuit are included in an implantable pulse generator.

18. The system of claim 9, including:
a user interface operatively coupled to the signal processing circuit;
wherein the sensing circuit is configured to sense an evoked potential signal for each electrode of multiple electrodes of the stimulation lead; and
wherein the signal processing circuit is configured to:
detect the changes in the sensed evoked potential signals; and
simultaneously present the changes of the sensed evoked potential signal for each electrode on the user interface.

19. A non-transitory computer readable storage medium including instructions that when performed by processing circuitry of a neurostimulation system, cause the neurostimulation system to perform actions including:
initiate delivery of neurostimulation to produce an evoked potential signal in a subject;
record sensed evoked potential signals resulting from the neurostimulation;
detect changes in the sensed evoked potential signals in response to a change in at least one of the number of repeated presentations of the neurostimulation or a change in a parameter of the first neurostimulation; and
display an indication of proximity of the stimulation lead to a preferred lead location according to the detected changes in the sensed evoked potential signals.

20. The non-transitory computer readable storage medium of claim 19, including instructions that cause the cause the neurostimulation system to perform actions including:
detect saturation in a sensed evoked potential signal and display an indication on the user interface that the stimulation lead is close to the preferred lead location in response to detecting the saturation;
detect when amplitude of a sensed evoked potential signal increases monotonically with the neurostimulation and display an indication on the user interface that the stimulation lead is off the preferred lead location in response to detecting the monotonical increase in the sensed evoked potential signal; and
detect when a rate of increase in amplitude of a sensed evoked potential signal changes and display an indication on the user interface that the stimulation lead is not close to the preferred lead location in response to detecting the change in rate of increase in amplitude.
